(19) United States
(12) Patent Application Publication
(10) Pub. No.: US 2002/0137674 A1
(43) Pub. Date: Sep. 26, 2002

(54) Method of using lectins for prevention and treatment of oral and alimentary tract disorders

(76) Inventors: Michael J. Oldham, Oxnard, CA (US); Howard C. Krivan, Santa Barbara, CA (US)

Correspondence Address: Vorys, Sater, Seymour and Pease LLP, Suite 1111, 1828 L Street, NW, Washington, DC, 20036-5104, US

(21) Appl. No.: 10/038,645

(22) Filed: Jan. 8, 2002

Related U.S. Application Data
(63) Continuation of application No. 08/861,596, filed on May 22, 1997, now abandoned.
Application No. 08/861,596 is a continuation of application No. 08/640,693, filed on May 1, 1996, now abandoned.
Application No. 08/640,693 is a continuation of application No. 08/385,306, filed on Feb. 7, 1995, now abandoned.

(51) Int. Cl. A61K 38/16; C07K 14/42

(52) U.S. Cl. 514/8; 530/395

(57) ABSTRACT

Infectious diseases caused by pathogenic microorganisms resident in the alimentary tract of humans and animals can be prevented and treated by administering to the alimentary tract of the human or animal an effective amount of a composition containing at least one lectin capable of binding to an infective microorganism and diminishing its infective capability of the microorganism. The lectin is administered dispersed in a pharmaceutically acceptable non-toxic vehicle.

### BACKGROUND OF THE INVENTION

1. Field of the Invention

This invention relates generally to methods of prevention and treatment of oral and alimentary diseases and more particularly to the use of oral administration of lectins for prophylaxis against and treatment of oral and alimentary diseases and disorders.

2. Brief Description of the Prior Art

Numerous diseases of humans and animals are caused by microorganisms that colonize the internal nasal passages and the alimentary tract, which comprises the mouth, pharynx, and gastrointestinal tract. While many of these diseases are acute conditions caused by bacteria that are self-limiting or treatable by conventional antibiotic therapy, others are caused by microorganisms that tend to establish chronic infections that cause continuing symptoms and are often difficult to treat with antibiotics.

Gastritis and duodenal peptic ulcers (commonly described as acid-peptic disease) involve an inflammation and/or erosion of the mucosal lining of the stomach or duodenum. These pathological conditions were thought for many years to be the result of hypersecretion of stomach acid caused by either genetic predisposition, stress, or diet, or a combination of these factors. This belief led to a medical treatment regime including drugs of various classes (antacids, histamine H₂ receptor antagonists, H⁺ inhibitors, K⁺ inhibitors, ATPase inhibitors and the like) that neutralize the excess acid or inhibit its secretion. While such therapy has had generally good results, it is often necessary to continue the treatment for the patient's entire lifespan because discontinuing treatment usually results in relapse of the disease. Recently, it has been established that the pathogen Helicobacter pylori, a spiral bacterium, is a factor in the development of gastritis and duodenal peptic ulcers. This bacterium has been found to colonize the gastric epithelium and to cause damage to the epithelial cells which results in a gastritis that predisposes the organ to the formation of ulcers. H. Pylori has also been linked to development of gastric adenocarcinoma and B cell lymphoma in the stomach. H. pylori's in vivo role in gastritis and peptic ulcers and its association with the fourth leading cause of cancer deaths in the world, gastric adenocarcinoma, make it one of the world's most prevalent and significant pathogens. There is no satisfactory antimicrobial agent known at present that is effective against H. pylori in vivo.

Cryptosporidium parvum is a pathogenic intestinal protozoan with worldwide distribution that is a frequent cause of both endemic and epidemic diarrheal illness. This illness is particularly devastating in immunocompromised individuals, producing diarrhea with profuse watery stools accompanied by cramping, abdominal pain, nausea, vomiting, malaise and low grade fever that increases over months and years. Currently, there are no preventative therapies and antiinfective drugs are of limited efficacy.

Periodontal disease is a major reason for tooth loss in adults. Microbiologically, periodontal disease is a polymicrobic problem involving anaerobic bacteria: Treponema denticola, Bacteroides forsythus, Actinobacillus actinomycetemcomitans, Campylobacter rectus, Prevotella intermedia, and Porphyromonas qingivalis, as well as others. This disease is more prominent in patients with dental implants, since the natural gum never fully adheres to the implant (false tooth) providing space for bacterial attachment and growth. Currently, treatments include more frequent tooth cleaning by dental hygienists, more frequent brushing with special dentifrices, and more frequent use of mouthwashes. While all current treatments decrease the probability and severity of periodontal disease, there is still a significant amount of tooth loss and none of the current approaches deals effectively with microbial attachment to the tooth or the buccal mucosa (gum).

Streptococcus pyogenes is an organism that can cause an acute pharyngitis with suppurative consequences caused by spread to other organs (otitis media, abscesses, meningitis, and the like) and/or non-suppurative consequences caused by toxins produced by some strains (scarlet fever). It is generally controllable with penicillins, but other methods of treatment are desirable because allergic reactions to penicillin are not uncommon.

Accordingly, a need has continued to exist for improved methods of treating and preventing disease of the oral cavity and alimentary tract caused by pathogenic microorganisms.

### SUMMARY OF THE INVENTION

This need for more convenient and effective therapy and prophylaxis of diseases of the nasal cavity and alimentary tract has now been alleviated by the method of this invention, according to which one or more lectins capable of binding to the surface of pathogenic microorganisms of the alimentary tract or nasal cavity or to the tissues that line the alimentary tract and nasal cavity them selves are administered orally or nasally to a patient infected with such pathogens or to a person in danger of being exposed to such pathogens.

Accordingly, it is an object of the invention to provide an improved method for treating acid-peptic disease.

A further object is to provide a method of prophylaxis for acid-peptic disease.

A further object is to provide a method of prophylaxis for gastritis.

A further object is to provide a method of treatment for gastritis.

A further object is to provide a method for prophylaxis against Cryptosporidium parvum.

A further object is to provide a method of treatment for infections caused by Cryptosporidium parvum.

A further object is to provide a method for prophylaxis against Streptococcus pyogenes.

A further object is to provide a method of treatment for infections caused by Streptococcus pyogenes.

A further object is to provide a method of prophylaxis for periodontal disease.

A further object is to provide a method of treating periodontal disease.

A further object is to provide a method for binding pathogenic microorganisms in the alimentary tract.

A further object is to provide a method for binding target cells in the alimentary tract.

A further object is to provide vehicles for delivering lectins to the alimentary tract.

Other objects of the invention will become apparent from the following detailed descriptions

### DETAILED DESCRIPTION OF THE INVENTION AND PREFERRED EMBODIMENTS

Lectins are carbohydrate-binding proteins of nonimmune origin that agglutinate cells or precipitate polysaccharides or glycoconjugates, i.e., proteins or lipids conjugated to oligo- or polysaccharides. They are widely distributed, and have been isolated from both plant and animal sources. Their reactions with living cells are based on their ability to bind with antibody-like specificity to particular arrangements of the sugar residues that make up oligo- or polysaccharides.

The surface of eucaryotic cells contains very numerous molecules of glycoproteins and glycolipids. Such glycoconjugates are found in the plasma membranes of cells of multicellular animals, including mammals and humans, as well as on the surfaces of single-celled eucaryotic organisms. Similarly, the cell walls of bacteria and the envelopes and capsids of viruses contain structural polysaccharides and/or glycoproteins. The carbohydrate moieties of these molecules which are displayed on the cell surfaces exhibit great variety in composition and structure that serves to distinguish the types of cells and to serve as a signal to other cells or materials which come into contact with the cell. For, example, variation in the carbohydrate moieties of glycoproteins in the plasma membrane of red blood cells serves as the basis for the conventional blood typing classification. When lectins recognize and bind to certain carbohydrate moieties they may serve to cross-link and agglutinate the cells bearing the binding groups, a property that earns for them the alternate name of agglutinins. Furthermore, because the same sort of carbohydrate moieties often serve as attachment points for pathogens to bind to target cells and invade them, lectins may block infection of target cells by blocking the sites used by pathogens as recognition markers. The same type of specific binding occurs between sperm and egg in conception, and can be blocked by lectins. The binding ability of lectins may be very specific for certain mono- or oligo-saccharides, allowing lectins to be used as a powerful tool for investigating the oligosaccharide epitopes on the surface of organisms or cells. Lectins can distinguish between blood cells of specific blood type, malignant from normal cells, and among species and genera of organisms. While glycoproteins, glycolipids, and bacterial cell walls and capsules are believed to be the main lectin-binding locations on the surfaces of cells, it is not excluded that carbohydrate moieties derived from other molecules or cellular structures may be displayed on the cell surface or that other lectin-binding structures may be targets for the lectins used in the method of this invention.

Current medical uses of lectins include distinguishing erythrocytes of different blood types (blood typing). More recently, lectins have been used ex-vivo in depleting T cells of patients undergoing bone marrow transplantation.

Among the microorganisms that are bound by certain lectins are infectious organisms such as bacterial protozoa, fungi, and viruses. Lectins may be used to identify such microorganisms in vitro and are also capable of binding to them in vivo, thereby preventing them from infecting living cells. Human disease-causing organisms (and the diseases caused by them) that can be bound by lectins include numerous sexually transmitted diseases as described in copending U.S. patent application Ser. No. 08/317,599, filed Oct. 3, 1994, as well as Helicobacter pylori, Cryptosporidium parvum, Treponema denticola; Bacteroides forsythus, Actinobacillus actinomycetemcomitans, Streptococcus pyogenes, Campylobacter rectus, Prevotella intermedia, and Porphyromonas gingivalis, as well as others . Other infections and diseases in which the portal of entry or initial attachment is nasal, oral, or in the alimentary tract are also capable of being prevented by administration of lectins according to this invention.

According to the invention, a dose of lectins effective to bind and agglutinate pathogenic microorganisms and/or block the recognition sites on target cells is administered to the nose, mouth, or alimentary tract prophylactically or as therapy. Because of the specificity of lectins for certain microorganisms, it is preferred to administer a mixture of lectins chosen for their properties of agglutinating specific pathogens.

A representative listing of lectins, the abbreviations by which they are referred to, and their sources is given in Table 1.

TABLE 1
Lectins and Abbreviations

| Lectin | Source |
|---|---|
| AAnA | Anguilla anguilla (Eel serum) |
| AAurA | Aleuria aurantia (Orange peel fungus) |
| ABA | Agaricus bisporus (Mushroom) |
| ABrA | Amphicarpanea bracteata (hog-peanut) |
| AL | Hippaestrum hybrid (Amaryllis bulbs) |
| APA | Abrus precatorius (Jequirity bean) |
| AS | Avena sativa (oat) |
| BDA | Bryonia dioica (white bryony) |
| BPA | Bauhinia purpurea alba (camel's foot tree) |
| CA | Colchicum autumnale (meadow saffron) |
| CAA | Caragana arborescens (Siberian pea tree) |
| CCA | Cancer antennarius (California crab) |
| ConA | Concanavalia ensiformis (Jack bean) |
| CPA | Cicer arietinum (chick pea) |
| CSA | Cytisus scoparius (Scotch broom) |
| DBA | Dolichos biflorus (horse gram) |
| DSA | Datura stramonium (Jimson weed, Thorn apple) |
| ECA | Erythrina crystagalli (Coral tree) |
| ECorA | Erythrina coralldendron (Coral tree) |
| EEA | Euonymus europaeus (spindle tree) |
| GNA | Galanthus nivalis (Snowdrop bulb) |
| GSA-1/GSA-1I | Griffonia simplicifolia |
| HAA | Helix aspersa (Garden snail) |
| HPA | Helix pomatia (Roman or edible snail) |
| JAC (Jacalin) | Artocarpus integrifolia (jackfruit) |
| LAA | Laburnum alpinum |
| LBA | Phaseolus lunatis (also limensis) (Lima bean) |
| LCA (LcH) | Lens culinaris (lentil) |
| LEA | Lycopersicon esculentum (Tomato) |
| LFA | Limax flavus (garden slug) |
| LOA | Lathyrus oderatus (Sweet pea) |
| LTA (LOTUS) | Lotus tetragonolobus (Asparagus pea) |
| MAA | Maackla amurensis (maackla) |
| MIH | Mangifera indica (Mango) |
| MPA | Maclura pomifera (Osage orange) |
| NPL (NPA) | Narcissus pseudonarcissus (daffodil) |
| PAA | Persea americana (Avocado) |
| PHA (PHA-L) | Phaseolis vulgaris (Red kidney bean) |
| PNA | Arachis hypogaea (Peanut) |
| PSA | Pisum sativum (Pea) |
| PWA | Phytolacca americana (pokeweed) |
| PTAgalactose | Psophocarpus tetagonolobus (winged bean) |
| PTAgalNac | Psophocarpus tetagonolobus (winged bean) |
| RCA-I/RCA-II | Ricinus communis (Castor bean) |
| RPA | Robinia pseudoaccacia (black locust) |
| SBA | Glycine max (Soybean) |
| SJA | Sophora japonica (Japanese pagoda tree) |
| SNA | Sambuccus nigra (elderberry) |
| STA | Solanium tuberosum (Potato) |
| TKA | Trichosanthes kinlowii (China gourd) |
| TL | Tulipa sp. (tulip) |
| TMT | Tomentine (seaweed Codium tomentosum) |
| UEA-I/UEA-I1 | Ulex europaeus (Gorse or Furz seeds) |
| VAA | Viscum album (European mistletoe) |
| VFA | Vicia faba (Fava bean) |
| VGA | Vicia graminea |
| VRA | Vigna radiata (mung bean) |
| VSA | Vicia sativa |
| VVA | Vicia villosa (Hairy vetch) |
| WFA | Wisteria floribunda (Japanese wisteria) |
| WGA | Triticum vulgaris (Wheat germ) |
| suc-WGA | Succinyl WGA |

The choice of lectins for prophylaxis or treatment of a particular infection is determined by the lectin-binding properties of the pathogenic microorganism, which is in turn determined by the composition of the particular oligosaccharide residues of the glycoproteins and glycolipids found on the external surface of the pathogen.

For example, Cryptosporidium parvum oocysts are bound by lectins that bind to N-acetyl-D-glucosamine residues on their surfaces (Llovo, J., et al., J. Infectious Diseases 1993, 167, pp. 1477-1480.). Such lectins include UEA-II and Tomentine. A lectin from Codium fragile (a type of seaweed) specific for N-acetyl-D-glucosamine also agglutinates Cryptosporidium parvum oocysts. Such lectins include BDA, ConA, BDA, SBA, GSA-I, GSA-II, HAA, HPA, LAA, LBA, RCA-II, SNA, SJA, and WGA.

A number of lectins can bind to oral mucosa and block potential attachment sites of pathogenic bacteria. Such lectins include DBA, LTA, RCA, SBA, UEA, and WGA.

While the lectins discussed above and the organisms against which they are effective are representative of useful lectins according to the invention, it is to be understood that other lectins may be discovered which are active in the binding and agglutination of nasal, oral and alimentary tract pathogens.

The selection of specific lectins to be administered will depend on the diseases sought to be prevented. It is preferred to administer a lectin or mixture of lectins, selected for best agglutinative efficacy against the specific pathogen or pathogens responsible for the disease. It is also according to the invention to prevent or treat infectious diseases caused by pathogenic microorganisms that colonize the surface of the mucosa lining the alimentary canal by administering a dose of lectins capable of binding to the receptors on the mucosal tissue to which the organisms bind in their attack on the mucosal cells. When the receptors on the cells are blocked, the initial binding of the microorganism to the cell, which in many cases is necessary for it to exert its pathological activity, is blocked, and the disease is prevented.

The lectins may be administered in any fluid or vehicle suitable for nasal or oral administration of pharmaceutical compounds. Inasmuch as lectins are generally dispersible in aqueous vehicles, the practitioner may choose a vehicle from among a broad range of conventional pharmaceutically acceptable non-toxic vehicles. Thus, mouthwash, chewing gum, pills, tablets (chewable and non-chewable), caplets, toothpaste, dental floss, nasal sprays, and the like, may be formulated in which the selected lectins are dispersed in a non-toxic vehicle for nasal, oral and alimentary tract administration.

A preferred embodiment of the invention comprises oral administration of lectins capable of binding to Helicobacter Pylori in order to prevent infection by that organism or to treat gastritis or duodenal ulcers related to infection with H. pylori. The treatment comprises administration to a patient infected with H. pylori an amount of a lectin capable of binding to H. pylori effective to diminish the infective capability of the microorganism. The exact dose will depend on the strength of binding between the lectin and H. pylori, i.e., on the binding constant of the interaction between the lectin and the receptors for the lectin on the surface of the microorganism, and on the number of surface receptors on the microorganism that have to be saturated with lectin in order to produce an effective decrease in the infective capability of the microorganism. The effective dose will also depend on the severity and extent of the infection, i.e., on the number of microorganisms present and the bioavailability of the lectin to interact with these micro-organisms and incapacitate their ability to bind to and injure the cells of the gastric and duodenal mucosa. Accordingly, while the practitioner can gain some guidance as to an effective dose from the experimental determination of the binding effectiveness of a given lectin for H. pylori, it must be expected that determination of an effective dose will involve some experimentation of the type that is entirely conventional in the development of pharmaceutical treatment of infectious diseases.

The practice of the invention will be illustrated by the following example which is intended to be illustrative and is not to be construed as limiting the scope of the appended claims.

### EXAMPLE

This example illustrates the binding of various lectins to Helicobacter pylori.

The efficacy of binding of several lectins to H. pylori was investigated in vitro by the following procedures.

Growth of bacteria: Toxigenic (ATCC 49503) and non-toxigenic (ATCC 43504, type strain) strains of H. pylori were obtained from the American Type Culture Collection (Rockville, Md.). H. pylori were grown under microaerophilic conditions at 37° C. for 4-5 days on blood agar plates containing 5% sheep blood. The bacteria were harvested with 0.01 M sodium phosphate buffer (pH 7.2) containing 0.15 M NaCl (PBS), washed twice and suspended to a final optical density of 0.15 in sodium bicarbonate buffer, pH 9.5, before being used.

Lectin Binding Assay: Biotinylated lectins were reconstituted in phosphate buffered saline (10 mM sodium phosphate−150 mM NaCl, pH 7.2) and stored in a freezer at −70° C. until used. Washed H. pylori were suspended in sodium bicarbonate buffer (pH 9.5). Microtiter plates washed with 95% ethanol and dried were coated with bacteria; by adding 200 μl of the suspension to each well and incubating overnight at room temperature. Wells coated with bacteria were washed three times with sodium acetate buffered saline, pH 4.0, containing 0.5% Tween 20 detergent (ABS-T), and the appropriate biotinylated lectin was added at the test concentration. Lectins defrosted at room temperature were diluted in each buffer, and 100 μl of various lectins was added to bacteria-coated wells at a final concentration of 50 μg/ml. After incubation in a humid chamber at room temperature for 2 hours, the wells were emptied and washed five times with ABS-T. Bound biotinylated lectin was detected by the addition of streptavidin-alkaline phosphatase (10 ng/μl) followed after two hours by washing three times with ABS-T and addition of 100 μl of freshly prepared p-nitrophenyl phosphate (1 mg/ml) in 0.1 M Tris buffer-0.15 M NaCl. Color production was quantitated by spectrophotometry at 405 nm.

The results of the lectin-binding tests are summarized in Table 2 for the toxigenic strain (ATCC 49503) and in Table 3 for the non-toxigenic strain (ATCC 43504). The tables present the following data:

1) Maximum rate of color production in the lectin binding assay (mOD/minute). This provides an indication of the maximum number of lectin binding sites.

2) Concentration of lectin which gives rise to 50% maximum rate of color production (micrograms/ milliliter). This provides an indication of the affinity of the binding sites.

3) Ratio (quotient) of maximum rate of lectin production to concentration of lectin at ½ the maximum rate.

In Tables 2 and 3 the first column indicates the lectin which was tested in the binding experiment, the numbers in the second and third columns are averages of the results of three replications of the lectin binding experiment with the indicated lectin, and the numbers in the third column represent the quotient of the average values given in the second and third columns.

TABLE 2
REACTIVITY OF PLANT LECTINS
WITH H. PYLORI (ATCC 49503)

| | Max. rate | [Lectin]_{½Max} | |
|---|---|---|---|
| Lectin | (mOD/min) | (μg/ml) | Quotient |
| sWGA | 188.37 | 0.63 | 299.00 |
| MPA | 358.63 | 1.56 | 229.89 |
| ConA | 273.92 | 1.54 | 177.87 |
| LEA | 295.81 | 2.06 | 143.60 |
| Jacalin | 332.96 | 3.26 | 102.13 |
| VVA | 529.35 | 4.80 | 110.28 |
| VFA | 518.79 | 5.45 | 95.19 |
| WGA | 3540.40 | 7.84 | 451.58 |
| CPA | 564.80 | 9.44 | 59.83 |
| WFA | 572.63 | 10.10 | 56.70 |
| LCA | 468.49 | 10.30 | 45.48 |
| GNA | 334.76 | 10.60 | 31.58 |
| NPA | 517.84 | 13.39 | 38.67 |
| TKA | 300.04 | 14.79 | 20.29 |
| STA | 300.16 | 14.82 | 20.25 |
| PSA | 185.44 | 14.93 | 12.42 |
| CSA | 655.79 | 15.88 | 41.30 |
| Lotus | 495.91 | 16.01 | 30.98 |
| MAA | 354.12 | 20.52 | 17.26 |
| LAA | 354.11 | 20.52 | 17.26 |
| SBA | 476.64 | 26.67 | 17.87 |
| BPA | 393.65 | 33.54 | 11.80 |
| LBA | 1425.53 | 34.05 | 41.87 |
| DSA | 241.72 | 55.01 | 4.39 |
| RPA | 281.01 | 71.77 | 3.92 |
| ABA | 125.44 | 115.82 | 1.08 |
| HAA | 467.62 | 147.15 | 3.18 |

TABLE 3
REACTIVITY OF PLANT LECTINS
WITH H. PYLORI (ATCC 43504)

| | Max. rate | [Lectin]_{½Max} | |
|---|---|---|---|
| Lectin | (mOD/min) | (μg/ml) | Quotient |
| sWGA | 93.56 | 0.43 | 217.58 |
| ConA | 177.18 | 1.06 | 167.15 |
| LCA | 377.36 | 2.11 | 178.84 |
| MPA | 411.39 | 2.12 | 194.05 |
| LEA | 418.61 | 2.60 | 161.00 |
| VFA | 240.90 | 2.84 | 84.82 |
| WGA | 869.79 | 3.03 | 287.06 |
| WFA | 660.37 | 3.15 | 209.64 |
| STA | 191.47 | 3.24 | 59.10 |
| LBA | 540.72 | 3.81 | 141.92 |
| VVA | 740.44 | 6.22 | 119.04 |
| NPA | 356.14 | 9.96 | 35.76 |
| CSA | 649.81 | 13.67 | 47.54 |
| Lotus | 463.49 | 27.91 | 16.79 |
| GNA | 298.92 | 17.63 | 16.96 |
| MAA | 392.32 | 22.61 | 17.35 |
| LAA | 390.01 | 25.70 | 15.18 |
| Lotus | 468.49 | 27.91 | 16.79 |
| SBA | 573.86 | 31.04 | 18.49 |
| ABA | 83.43 | 38.87 | 2.15 |
| TKA | 657.29 | 54.91 | 11.97 |
| BPA | 596.88 | 55.30 | 10.79 |
| JAC | 337.65 | 66.96 | 5.04 |
| RPA | 658.70 | 84.81 | 7.77 |
| DSA | 315.7 | 113.25 | 2.79 |
| HAA | 685.63 | 324.93 | 2.11 |

In these assays, the numbers representing the concentration of lectin which gives rise to 50% maximum rate of color production provide a measure of the ability of each lectin to bind to H. pylori and thereby of its potential usefulness in prophylaxis against infections by H. pylori and treatment of such infections. The smaller values represent a greater affinity and hence a greater usefulness in prophylaxis and therapy. In practice, those lectins having a value of [lectin]_{½max} greater than about 50 are not expected to be useful as agents against H. pylori. Those lectins having a value of [lectin]_{½max} less than about 8.00 have especially good binding properties with regard to H. pylori and are expected to be particularly useful in prophylaxis and therapy. Such preferred lectins include sWGA, MPA, ConA, LEA, Jacalin, VVA, VFA and WGA.

The invention having now been fully described, it should be understood that it may be embodied in other specific forms or variations without departing from its spirit or essential characteristics. Accordingly, the embodiments described above are to be considered in all respects as illustrative and not restrictive, the scope of the invention being indicated by the appended claims rather than by the foregoing description, and all changes which come within the meaning and range of equivalency of the claims are intended to be embraced therein.

We claim:

1. A method of preventing and/or treating infectious diseases caused by pathogenic microorganisms resident in the alimentary tract or nasal cavity of humans and animals comprising administering to the alimentary tract or nasal cavity of a human or animal an amount of a composition containing at least one lectin capable of binding to an infective microorganism resident in said alimentary tract or nasal cavity, said lectin being effective to diminish the infective capability of said microorganism, said lectin being dispersed in a pharmaceutically acceptable non-toxic vehicle.

2. The method of claim 1 wherein a plurality of said lectins is administered.

3. The method of claim 1 wherein said microorganism is Helicobacter pylori.

4. The method of claim 2 wherein said microorganism is Helicobacter pylori.

5. The method of claim 3 wherein said lectin is selected from the group consisting of sWGA, MPA, ConA, LEA, Jacalin, VVA, VFA, WGA, CPA, WFA, LCA, GNA, NPA, TKA, STA, PSA, CSA, Lotus, MAA, LAA, SBA, BPA, and LBA.

6. The method of claim 5 wherein said lectin is selected from the group consisting of sWGA, MPA, ConA, LEA, Jacalin, VVA, VFA, WGA, CPA, WFA, LCA, GNA, NPA, TKA, STA, PSA, CSA, Lotus, MAA, LAA, SBA, BPA, and LBA.

7. The method of claim 4 wherein said lectin is selected from the group consisting of sWGA, MPA, ConA, LEA, Jacalin, VVA, VFA, WGA, CPA, WFA, LCA, GNA, NPA, TKA, STA, PSA, CSA, Lotus, MAA, LAA, SBA, BPA, and LBA.

8. The method of claim 7 wherein said lectin is selected from the group consisting of sWGA, MPA, ConA, LEA, Jacalin, VVA, VFA, WGA, CPA, WFA, LCA, GNA, NPA, TKA, STA, PSA, CSA, Lotus, MAA, LAA, SBA, BPA, and LBA.

9. The method of claim 1 wherein said microorganism is Cryptosporidium parvum.

10. The method of claim 2 wherein said microorganism is Cryptosporidium parvum.

11. The method of claim 1 wherein said microorganism is selected from the group consisting of Treponema denticola, Bacteroides forsythus, Campylobacter rectus, Prevotella intermedia, Porphyromonas gingivalis, and species of Actinobacillus actinomycetemcomitans.

12. The method of claim 2 wherein said microorganism is selected from the group consisting of Treponema denticola, Bacteroides forsythus, Campylobacter rectus, Prevotella intermedia, Porphyromonas gingivalis, and species of Actinobacillus actinomycetemcomitans.

13. The method of claim 1 wherein said microorganism is Streptococcus pyogenes.

14. The method of claim 2 wherein said microorganism is Streptococcus pyogenes.

15. The method of claim 1 wherein said lectin is capable of binding to the oral mucosa and is administered to the oral mucosa.

16. The method of claim 15 wherein said lectin is selected from the group consisting of DBA, LTA, RCA, SBA, UEA, and WGA.
